(19) United States
(12) Patent Application Publication
(10) Pub. No.: US 2014/0330263 A1
(43) Pub. Date: Nov. 6, 2014

(54) PERICARDIAL CUTTING ASSEMBLY

(71) Applicant: Didier De Canniere, Miami Beach, FL (US)

(72) Inventor: Didier De Canniere, Miami Beach, FL (US)

(21) Appl. No.: 13/875,664

(22) Filed: May 2, 2013

(51) Int. Cl. A61B 17/32; A61B 18/08

(52) U.S. Cl. CPC A61B 17/320016; A61B 18/082
USPC 606/28; 606/170

(57) ABSTRACT

The present invention is directed to an assembly, and methods thereof, for cutting the pericardium of a subject. The assembly is designed for insertion into the thoracic cavity via a thoracic passage localized between adjacent ribs of the subject. The assembly is particularly useful for cutting a window in the pericardium to allow for access to the heart for performing further procedures.

### STATEMENT REGARDING FEDERALLY-SPONSORED RESEARCH AND DEVELOPMENT

Not applicable.

### BACKGROUND OF THE INVENTION

1. Field of the Invention

This invention relates generally to the field of surgical devices. More particularly, this invention relates to an assembly for insertion into a thoracic passage for cutting of the pericardium.

2. Description of Related Art

Intracardiac intervention is often necessary to correct or repair cardiac components damaged by disease. Historically, such repair is accomplished by performing surgery characterized by the chest of the patient being opened to gain access to the heart. Often, such procedures further necessitate the heart being arrested and/or bypassed in order to perform the necessary procedures. Such procedures pose many possibilities for complications resulting in life altering consequences or even death.

Alternatively, it is now common for surgeons to introduce catheters into blood vessels to perform procedures at or near the heart. However, there are practical limitations to such procedures because of the limited size and/or layout of a patient's arteries. Often, catheters must be of a smaller caliber and require intricate maneuverability to reach target areas. Furthermore, the distance that a catheter must travel in some instances can be a drawback because the longer distance means more chance of puncturing blood vessel walls and/or increasing maneuverability issues.

Heart disease is the leading cause of death in the United States. Therefore, this further necessitates improved approaches to intracardiac intervention. There is a particular need for procedures and devices that would not require opening of the entire chest cavity and/or arrest of the heart to gain access to the chambers of the heart. Furthermore, there is a need for devices and procedures that provide more localized insertion sites for catheters to gain access to the heart so that the aforementioned risks and complications are reduced.

### SUMMARY OF THE INVENTION

The present invention addresses the need for an apparatus and/or assembly that facilitates the ability to perform surgical procedures on the heart without requiring opening of the entire thoracic cavity or catheters inserted at distal regions from the heart. As such, the present invention is directed in at least one embodiment to an assembly for insertion through a thoracic passage for cutting the pericardium. The assembly is particularly useful for cutting a window in the pericardium to allow for access to the heart muscle for performing further procedures.

The assembly includes a central shaft which includes an operating end and an engagement end. The operating end of the central shaft may also include an end structure configured to facilitate extension and retraction of the central shaft within the thoracic passage. The assembly also includes an elongated sheath which has a proximal end and a distal end. The elongated sheath is disposed on the central shaft between the operating end and the engagement end and in a movable and/or rotatable disposition relative to the central shaft.

The assembly further includes a blade instrument movably engaged with the elongated sheath. The blade instrument is movable into either a retracted orientation or a cutting orientation. The cutting orientation includes the blade instrument being extended in an operative fashion at the distal end of the elongated sheath. In the cutting orientation, the blade instrument is dimensioned and structured to cut in a cutting path, which may be in a curvilinear motion. In some embodiments, the blade cuts in a substantially circular motion. The central shaft is further dimensioned and configured at the engagement end to engage the pericardium and facilitate placement of the pericardium in a position for the blade instrument to cut the pericardium when the blade instrument is in the cutting orientation. The retracted orientation of the blade instrument is characterized by being structured to facilitate passage of the assembly through the thoracic passage. In some embodiments, the blade instrument may be further configured to include a blade handle movably engaged with the elongated sheath and structured to facilitate movement of the blade instrument between the retracted orientation and the cutting orientation.

The engagement end of the central shaft may contain various structures. In some embodiments, the engagement end of the central shaft may comprise a hook structure operative to engage the pericardium. In other embodiments, the engagement end may be in communication with a vacuum source and structured to exert a securing negative pressure on the exterior of the pericardium.

In further embodiments, the assembly may include, or be used in conjunction with, a port sheath having a predetermined intrathoracic length. The port sheath may be structured and dimensioned to receive and facilitate passage therethrough of the assembly into the thoracic cavity. Furthermore, at least a portion of the port sheath may be formed of a flexible material, where the flexible material is structured to demonstrate sufficient flexibility to accommodate movement between the pericardium and the thoracic passage when the assembly is inserted into the thoracic cavity via the port sheath.

In yet another embodiment, the blade instrument may be connected in communication with a cauterization source. The cauterizing source may be structured in conjunction with the blade instrument to exert pulses of heat on the exterior of the pericardium sufficient to cauterize the incision when the blade instrument is in the cutting orientation.

Also, at least a portion of the blade instrument may be formed of a flexible material such as, but not limited to, metal. The flexible material may be structured to demonstrate sufficient flexibility to accommodate movement of the blade instrument between the retracted orientation and the cutting orientation. As such, the flexible material of the blade instrument is structured to have an inherent bias that facilitates proper movement and positioning of the blade instrument to assume the cutting orientation when it is retracted from within the elongated sheath.

The present invention is also directed to a method of cutting a window in the pericardium of a subject. The method includes creating a passage into the thoracic cavity; obtaining a pericardial cutting assembly; inserting the pericardial cutting assembly into the thoracic cavity in communicating relation with the pericardium of the subject; and cutting the pericardium such that a window is created in the pericardium. In further embodiments, the method also includes pulling the pericardium with the pericardial cutting assembly prior to cutting the window. Also, the window may be cut in a cutting path, which may be in a curvilinear motion, based on the movement of the pericardial cutting assembly.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a perspective view of one embodiment of an assembly of the present invention.

FIG. 2A is a longitudinal, cross-sectional view of an assembly of the present invention.

FIG. 2B is a perspective view of an embodiment of a blade instrument prior to incorporation into an assembly of the present invention.

FIG. 3 is a perspective view of an embodiment of an assembly of the present invention inserted into a port sheath.

FIG. 4 is a perspective view of an embodiment of an assembly of the present invention in the cutting orientation.

Like reference numerals refer to like parts throughout the several views of the drawings.

### DETAILED DESCRIPTION OF THE PREFERRED EMBODIMENT

The present invention is directed to an assembly for insertion through a thoracic passage for cutting the pericardium. The assembly is particularly useful for cutting a window in the pericardium to allow for access to the heart muscle and/or allow fluid to drain from the pericardium in cases where the tissue layers surrounding the heart are inflamed (e.g., pericarditis).

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting of the invention. As used herein, the indefinite articles “a”, “an” and “the” should be understood to include plural reference unless the context clearly indicates otherwise.

The phrase “and/or,” as used herein, should be understood to mean “either or both” of the elements so conjoined, i.e., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the term “comprising.”

As used herein, the term “subject” refers to an animal. Typically, the terms “subject” and “patient” may be used interchangeably herein in reference to a subject. As such, a “subject” includes a human that is being treated as a patient.

The term “animal,” includes, but is not limited to, mouse, rat, dog, cat, rabbit, pig, monkey, chimpanzee, and human.

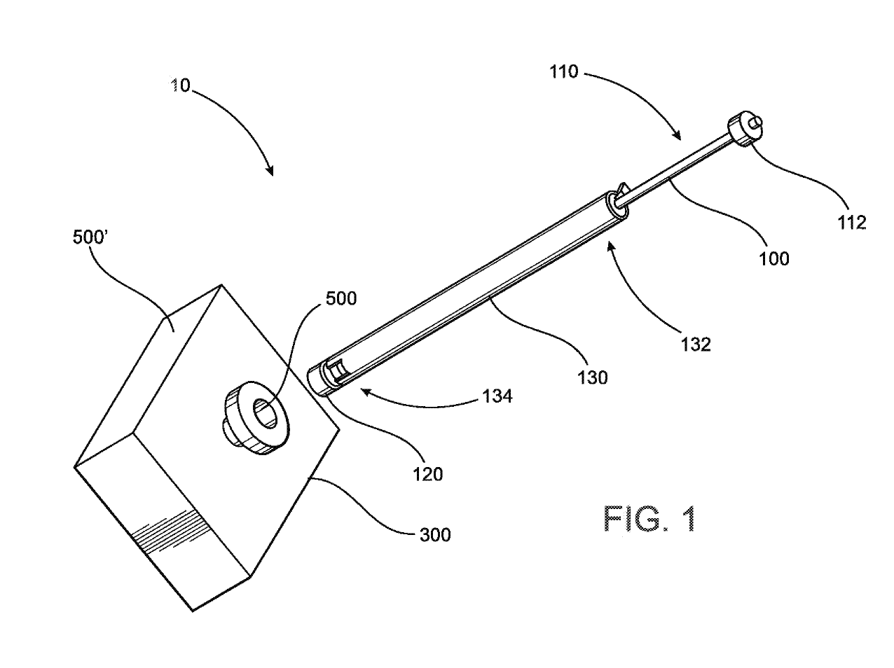

As illustrated in the accompanying drawings, and with primary reference to FIG. 1, the present invention is directed to an assembly, generally indicated as 10, for insertion through a thoracic passage 500. As will be set forth in greater detail hereinafter, the assembly 10 passes through the thoracic wall 500′ via the thoracic passage 500 through an intercostal space between appropriately positioned ribs to interact with and cut the pericardium. The assembly 10 is particularly useful for cutting a window in the pericardium to allow for access to the heart muscle to perform further cardiac procedures and/or to allow fluid to drain from the pericardium in cases where the tissue layers surrounding the heart are inflamed (e.g., pericarditis).

The assembly 10 includes a central shaft 100 which includes an operating end 110 and an engagement end 120. The operating end 110 of the central shaft 100 may also include an end structure 112 configured to facilitate extension and retraction of the central shaft 100 within the thoracic passage 500. Also, the end structure 112 may be any type of attachment that facilitates the movement and/or rotation of the central shaft 100 by the user to aid in the pulling and manipulation of the pericardial wall.

The assembly 10 also includes an elongated sheath 130 which has a proximal end 132 and a distal end 134. The elongated sheath 130 is disposed on the central shaft 100 between the operating end 110 and the engagement end 120 and in a movable and/or rotatable disposition. The elongated sheath 130 and central shaft 100 are capable of sliding and rotating relative to one another. Generally, the elongated sheath 130 has a diameter that corresponds to the diameter of the incision in the thoracic wall 500′ or the diameter of any cannula or the like (e.g., a Thoracoport™) that is used to guide the assembly 10 through the thoracic passage 500 into the thoracic cavity.

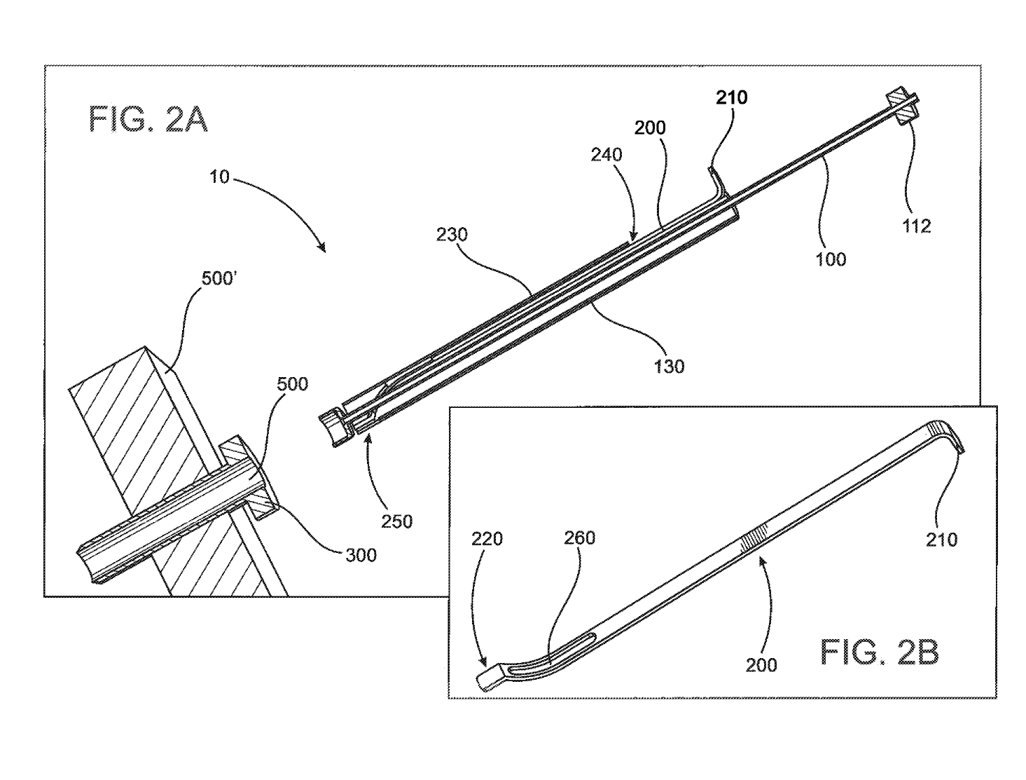

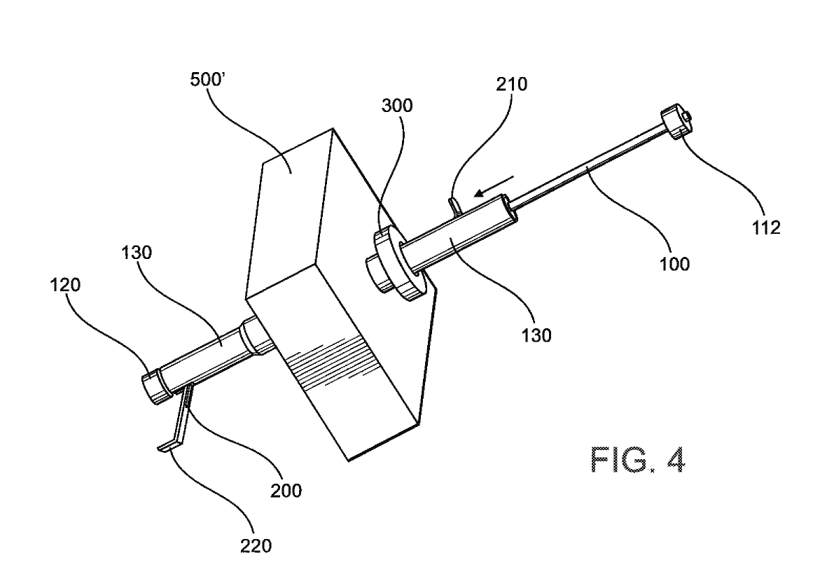

The assembly 10 further includes a blade instrument 200 movably engaged within the elongated sheath 130. FIG. 2B illustrates an embodiment of a blade instrument 200 that is useful when incorporated into the assembly 10. The blade instrument 200 is movable into either a retracted orientation (as illustrated in FIGS. 1, 2A, and 3) or a cutting orientation (as illustrated in FIG. 4). The cutting orientation includes the blade instrument 200 being extended in an operative fashion at the distal end 134 of the elongated sheath 130 while the retracted orientation maintains the blade instrument 200 within the elongated sheath 130 such that there is no ability for the cutting end 220 of the blade instrument 200 to inadvertently cut surrounding tissues while being inserted through the thoracic passage 500 into the thoracic cavity.

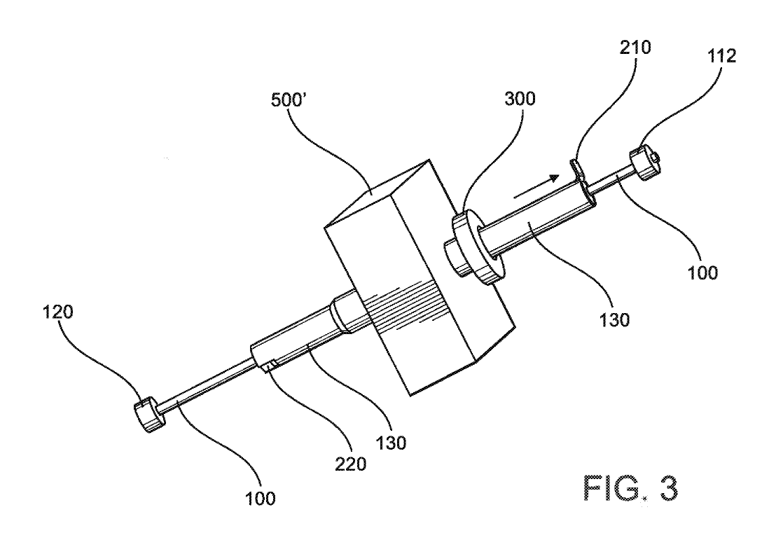

FIG. 3 illustrates the assembly 10 inserted into the thoracic cavity with the central shaft 100 extended into the cavity for contact with the pericardial wall. The blade instrument 200 of the assembly 10 illustrated in FIG. 3 is in the retracted orientation. The retracted orientation of the blade instrument 200 is characterized by being structured to facilitate passage of the assembly 10 through a thoracic passage 500, as shown, without inadvertent cuts to surrounding tissue(s).

As illustrated in FIG. 4, the blade instrument 200 is dimensioned and structured to cut in a cutting path, which may be in a curvilinear motion, when in the cutting orientation. The central shaft 100 is further dimensioned and configured at the engagement end 120 to engage the pericardium and facilitate placement of the pericardium in a position for the blade instrument 200 to cut the pericardium when the blade instrument 200 is in the cutting orientation.

As illustrated in FIGS. 2A and 2B, the blade instrument 200 may be further configured to include a blade handle 210 movably engaged with the elongated sheath 130 and structured to facilitate movement of the blade instrument 200 between the retracted orientation and the cutting orientation. As shown in FIG. 3, the blade handle 210 may be oriented closer to the proximal end 132 of the elongated sheath 130 when in the retracted orientation. Furthermore, as shown in FIG. 4, the blade handle 210 may be oriented in such a way that handle 210 is positioned toward the distal end 134 when the blade instrument 200 is in the cutting orientation. As such, a user may push the blade handle 210 in a direction toward the distal end 134 of the elongated sheath 130 to expose the cutting end 220 of the blade instrument 200 (as depicted in FIG. 4) and perform the reverse procedure to retract the cutting end 220 (as depicted in FIG. 3).

In addition to the movements of the blade instrument 200 between the cutting and retracted orientations, the central shaft 100 is utilized to effectively pull the pericardial sac upward and in position, or otherwise maintain positioning of the pericardial sac, for the cutting end 220 of the blade instrument 200 to create the window in the pericardium. As such, the assembly 10 contains a combination of moving parts that work in concert. The central shaft 100 moves up and down through the elongated sheath 130 while the elongated sheath 130 rotates around the central shaft 100 to enable the circular cutting motion of the blade instrument 200. Also, the blade instrument 200 moves upward and downward within the elongated sheath 130 to expose or retract the cutting end 220 of the blade instrument 200.

The engagement end 120 of the central shaft 100 may contain various structures that enable “grasping” of the pericardial sac. In some embodiments, the engagement end 120 of the central shaft 100 may comprise a screw or hook structure operative to engage the pericardium. In other embodiments, the engagement end 120 may be in communication with a vacuum or negative pressure source and structured to exert a securing negative pressure on the exterior of the pericardium. The engagement end 120 may be connected to the vacuum source preferably through one or more lumens, flow lines, conduits or other appropriate structures connected to or mounted on the assembly 10. As such, fluid communication is established between the engagement end 120 and the vacuum source to the extent that and appropriate negative pressure is developed and communicated to the engagement end 120 through the flow lines. The negative pressure is sufficient to removably secure the engagement end 120 to the exterior surface of the pericardial bag in appropriately adjacent relation to the entry site (window) formed in the pericardial bag.

In further embodiments, the assembly 10 may include, or be used in conjunction with, a port sheath 300 (i.e., introductory assembly) such as, but not limited to, the assemblies disclosed in U.S. patent application Ser. Nos. 13/570,347 and/or 13/442,230, by the inventor herein. The port sheath 300 may have a predetermined intrathoracic length and be structured and dimensioned to receive and facilitate passage therethrough of the assembly 10 into the thoracic passage 500. A port sheath 300 is illustrated in FIGS. 1, 2A, 3 and 4 being utilized with the assembly 10; however, it would be understood by those skilled in the art that the procedure could be performed without such a structure or with similar structures designed to provide a port between the exterior and interior of the thoracic wall 500′. Structures that may be useful as a port sheath 300 include, but are not limited to, cannulas such as a Theracoport™.

The fragile nature of the heart and the possibility of relative displacement or movement between the pericardial bag and the heart is recognized in the medical arts. Accordingly, additional preferred embodiments of the present invention include at least a portion of the port sheath 300 being formed of a flexible material. The flexible material portion of the port sheath 300 should be structured to demonstrate sufficient and/or a predetermined minimum amount of flexibility to accommodate movement between the pericardial bag and the heart relative to the thoracic passage 500 when the assembly 10 is inserted into the thoracic cavity via the port sheath 300. Such flexibility should help minimize or eliminate the possibility of tearing or inadvertently cutting the heart or surrounding tissues.

In yet another embodiment, the blade instrument 200 may be connected in communication with a cauterization source. The cauterizing source may be structured in conjunction with the blade instrument 200 to exert pulses of heat on the exterior of the pericardium sufficient to cauterize the incision when the blade instrument 200 is in the cutting orientation. The blade instrument 200 may be connected to the cauterization source preferably through one or more lumens, electrically conductive lines, conduits or other appropriate structures connected to or mounted on the assembly 10. As such, heating communication is established between the blade instrument 200 and the cauterization source to the extent that appropriate heat is developed and communicated to the cutting end 220 of the blade instrument 200 through the lines to cauterize the tissues of the pericardium as they are being cut.

Also, at least a portion of the blade instrument 200 may be formed of a flexible material such as, but not limited to, a flexible metal. The flexible material may be structured to demonstrate sufficient flexibility to accommodate movement of the blade instrument between the retracted orientation and the cutting orientation. As such, the flexibility of the material of the blade instrument 200 is sufficient to demonstrate an inherent bias, at least when the blade is in the retracted orientation, such as within the elongated sheath 130. As such, when in the retracted orientation within the elongated sheath 130, the blade instrument 200 is normally biased to move outwardly from the retracted orientation, within the elongated sheath 130, into the cutting orientation. The material may be, but is not limited to, any type of metal, metal alloy or other appropriate material having sufficient flexibility and the ability to be formed with a sharp edge for cutting at the cutting end 220. Furthermore, some embodiments require that the flexible material have conductive properties to facilitate cauterization of cut tissue.

Another aspect of the elongated sheath 130 is that some embodiments may contain an internal groove 230 that spans substantially from the distal 134 and proximal 132 ends, as illustrated in FIG. 2A. The internal groove 230 is configured such that the blade instrument 200 can slideably engage the internal groove 230 within the elongated sheath 130. The internal groove 230 may be further configured such that the groove extends substantially from at or near the proximal end 132 at an entry channel 240 to at or near the distal end 134 at an exit channel 250 wherein the groove 230 curves near the distal end 134 to position the exit channel 250 on an opposite side of the elongated sheath 130 from the entry channel 240, as shown in FIG. 2A. The exit channel 250 may be further adapted to substantially surround the cutting end 220 of the blade instrument 200 when in the retracted orientation.

Furthermore, in order to provide movement of the blade instrument 200 within the internal groove 230 in such a way that the central shaft 100 does not obstruct such movement, the blade instrument 200 may contain a cutout region 260 at or near the cutting end 220 of the blade instrument 200, as illustrated in FIG. 2B. In other embodiments, a cutout region may be at other positions, or on multiple positions, on the blade instrument 200 to accommodate the positioning of the blade instrument 200 with respect to the positioning of the entry channel 240 of the internal groove 230 and the central shaft 100 relative to the exit channel 250. The cutout region 260 is disposed, dimensioned and configured to facilitate passage of at least a portion of the central shaft 100 there through, such as when the bade instrument 200 moves between the retracted an cutting orientations. Therefore the cutout region 260 prevents the central shaft 100 from affecting or interfering with the biased orientation of the blade instrument 200 when in the cutting orientation. Likewise, the cutout region 260 is further dimensioned and positioned such that it allows the blade instrument 200 to slide within the internal groove 230 into the retracted orientation unobstructed by the central shaft 100.

The present invention is also directed to a method of cutting a window in the pericardium of a subject. Accordingly, in cooperation with the assembly as set forth herein, the method comprises creating a passage into the thoracic cavity and inserting the pericardial cutting assembly into the thoracic cavity in communicating relation with the pericardium of the subject. The assembly is inserted with the blade instrument in the retracted orientation into the thoracic cavity via the thoracic passage that may include a port sheath as described herein to aid in positioning of the assembly. The assembly is further utilized with the blade instrument engaged in the cutting orientation to cut the pericardium such that a window is created in the pericardium. In further embodiments, the method also includes pulling the pericardium with the engagement end of the central shaft of the pericardial cutting assembly to position the pericardium prior to cutting the window. The pulling of the pericardium can occur, depending on the configuration of the engagement end, by use of screw(s), hook(s) or a vacuum (as described previously herein). Also, the window may be cut in a cutting path, which may be in a curvilinear motion, (e.g., circular motion) based on the movement of the pericardial cutting assembly. The method may also include cauterization of the incision in the pericardium via a cauterization source as previously described herein.

Without further elaboration, it is believed that one skilled in the art can, using the preceding description, utilize the present invention to its fullest extent. Any one or more of the features of the previously described embodiments can be combined in any manner with one or more features of any other embodiments in the present invention. Furthermore, many variations of the invention will become apparent to those skilled in the art upon review of the specification.

It is to be appreciated that the foregoing Detailed Description section, and not the Abstract section, is intended to be used to interpret the claims. The Abstract section may set forth one or more, but not all, exemplary embodiments of the present invention as contemplated by the inventor(s), and thus, is not intended to limit the present invention and the appended claims in any way.

The foregoing description of the specific embodiments should fully reveal the general nature of the invention so that others can, by applying knowledge within the skill of the art, readily modify and/or adapt for various applications such specific embodiments, without undue experimentation, without departing from the general concept of the present invention. Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents. Moreover, the breadth and scope of the present invention should not be limited by any of the above-described exemplary embodiments, but should similarly be defined only in accordance with the following claims and their equivalents.

Now that the invention has been described,

What is claimed is:

1. An assembly for insertion through a thoracic passage and for cutting the pericardium, said assembly comprising:
a central shaft including an operating end and an engagement end;
an elongated sheath including a proximal end and a distal end, said elongated sheath movably and rotatably disposed on said central shaft between said operating end and said engagement end;
a blade instrument movably engaged within said elongated sheath and movable into and out of a cutting orientation at the distal end of said elongated sheath, said blade instrument dimensioned and structured to cut in a cutting path when in the cutting orientation;
said central shaft dimensioned and configured at said engagement end to engage the pericardium and facilitate placement of the pericardium in a position for said blade instrument to cut the pericardium when said blade instrument is in the cutting orientation.

2. The assembly of claim 1 wherein said engagement end of said central shaft comprises a hook structure operative to engage the pericardium.

3. The assembly of claim 1 wherein said blade instrument is dimensioned and structured to cut in a circular cutting path when in the cutting orientation.

4. The assembly of claim 1 further comprising a vacuum source; said engagement end connected in communication with said vacuum source and structured to exert a securing negative pressure on the exterior of the pericardium.

5. The assembly of claim 1 further comprising a port sheath having a predetermined intrathoracic length, said port sheath structured and dimensioned to receive and facilitate passage therethrough of the assembly into the thoracic cavity.

6. The assembly of claim 5 wherein said blade instrument is disposable between a retracted orientation and the cutting orientation, the retracted orientation of said blade instrument being structured to facilitate passage of said central shaft and said elongated sheath through said port sheath.

7. The assembly of claim 5 wherein at least a portion of said port sheath is formed of a flexible material, said flexible material structured to demonstrate sufficient flexibility to accommodate movement between the pericardium and the thoracic passage.

8. The assembly of claim 5 wherein said blade instrument is configured to include a blade handle movably engaged with said elongated sheath and structured to facilitate movement of the blade instrument between the retracted orientation and the cutting orientation.

9. The assembly of claim 1 wherein said blade instrument is disposable between a retracted orientation and the cutting orientation, the retracted orientation of said blade instrument being structured to facilitate passage of said central shaft and said elongated sheath through the thoracic passage.

10. The assembly of claim 1 wherein said operating end of said central shaft comprises an end structure configured to facilitate extension and retraction of said central shaft within the thoracic passage.

11. The assembly of claim 1 wherein said blade instrument is configured to include a blade handle movably engaged with said elongated sheath and structured to facilitate movement of the blade instrument between the retracted orientation and the cutting orientation.

12. The assembly of claim 1 further comprising a cauterization source; said blade instrument connected in communication with said cauterization source and structured to exert pulses of heat on the exterior of the pericardium sufficient to cauterize the incision when in the cutting orientation.

13. The assembly of claim 1 wherein at least a portion of said blade instrument is formed of a flexible metal, said flexible metal structured to demonstrate sufficient flexibility to accommodate movement of the blade instrument between the retracted orientation and the cutting orientation.

14. A method of cutting a window in the pericardium of a subject, said method comprising:
creating a passage into the thoracic cavity;
obtaining a pericardial cutting assembly;
inserting the pericardial cutting assembly into the thoracic cavity in communicating relation with the pericardium of the subject;
cutting the pericardium, whereby a window is created.

15. The method of claim 14, further comprising pulling the pericardium with the pericardial cutting assembly prior to cutting.

16. The method of claim 14, wherein cutting the pericardium is performed in a curvilinear motion.
